(19) United States
(12) Patent Application Publication
(10) Pub. No.: US 2007/0027101 A1
(43) Pub. Date: Feb. 1, 2007

(54) Methods for treating ocular neovascular diseases

(76) Inventors: David R. Guyer, New York, NY (US); Anthony P. Adamis, Bronxville, NY (US); Denis O'Shaughnessy, Mendham, NJ (US)

Correspondence Address: (OSI) EYETECH, INC., 3 TIMES SQUARE 12TH FLOOR, NEW YORK, NY, 10036, US

(21) Appl. No.: 11/491,818

(22) Filed: Jul. 24, 2006

Related U.S. Application Data
(63) Continuation of application No. 10/786,491, filed on Feb. 25, 2004, now abandoned.
Application No. 10/786,491 is a continuation of application No. 10/291,091, filed on Nov. 8, 2002.
(60) Provisional application No. 60/332,304, filed on Nov. 9, 2001.

(51) Int. Cl. A61K 48/00

(52) U.S. Cl. 514/44

(57) ABSTRACT

Disclosed herein are methods for treating ocular neovascular disease using anti-VEGF therapy in combination with a second therapy that inhibits the development of ocular neovascularization or destroys abnormal blood vessels in the eye, such as photodynamic therapy.

### CROSS REFERENCE TO RELATED APPLICATIONS

This application is a continuation of U.S. application Ser. No. 10/291,091, filed Nov. 8, 2002, which claims priority to U.S. Provisional Application Ser. No. 60/332,304, filed on Nov. 9, 2001.

### FIELD OF THE INVENTION

The invention relates to methods for treating ocular neovascularization using agents that inhibit VEGF.

### BACKGROUND OF THE INVENTION

Angiogenesis, or abnormal blood vessel growth, has been implicated as an important cause of pathological states in many areas of medicine, including ophthalmology, cancer, and rheumatology. For example, the exudative or neovascular form of age-related macular degeneration (AMD) is a leading cause of blindness in the elderly. There is currently no standard and effective therapy for the treatment of exudative ADM in most patients. Thermal laser photocoagulation and photodynamic therapy (PDT) have been shown to be beneficial for subgroups of such patients. However, only a fraction of eyes meet the eligibility criteria for such therapeutic interventions and those treated have a high recurrence rate.

Recent pre-clinical studies have suggested that pharmacological intervention or anti-angiogenesis therapy may be useful to treat various forms of ocular neovascularization, such as choroidal neovascularization (CNV). Much of this work has focused on blocking vascular endothelial growth factor (VEGF), which has been implicated in the pathogenesis of CNV secondary to AMD and the pathogenesis of diabetic retinopathy. VEGF is an important cytokine growth factor involved in angiogenesis and appears to play a critical role in the development of ocular neovascularization. Human studies have shown that high concentrations of VEGF are present in the vitreous in angiogenic retinal disorders but not in inactive or non-neovascularization disease states. Excised human CNV after experimental submacular surgery have also shown high VEGF levels. Other studies have shown regression or prevention of neovascularization in multiple vascular beds in several animal models, using various types of anti-VEGF agents, including antibody fragments. Thus, anti-VEGF therapy is a promising new treatment for AMD, diabetic retinopathy, and related disorders.

In addition to a potential anti-angiogenic effect, anti-VEGF therapy may be useful as an anti-permeability agent. VEGF was initially referred to as vascular permeability factor due to its potent ability to induce leakage from blood vessels. Recent research has shown that VEGF may be important in causing vessel leakage in diabetic retinopathy and that the diabetes-induced blood-retinal barrier breakdown can be dose-dependently inhibited with anti-VEGF therapy. Anti-VEGF therapy may, therefore, represent a two-prong attack on CNV via its anti-angiogenic and anti-permeability properties.

Existing methods for treating ocular neovascular disease are in need of improvement in their ability to inhibit or eliminate various forms of neovascularization, including choroidal neovascularization secondary to AMD and diabetic retinopathy. Furthermore, there is a continuing and significant need to identify new therapies to treat ocular neovascularization. The present invention fulfills these needs and further provides other related advantages.

### SUMMARY OF THE INVENTION

We have conducted clinical trials of an anti-VEGF aptamer with and without photodynamic therapy in patients with subfoveal choroidal neovascularization secondary to age-related macular degeneration to determine the safety profile of multiple injection therapy. We found that anti-VEGF therapy with or without photodynamic therapy (PDT) was both safe and effective in treating patients suffering from AMD and related disorders. Most patients receiving the anti-VEGF aptamer exhibited stable or improved vision three months after treatment. Those receiving anti-VEGF therapy in combination with PDT exhibited the most dramatic improvement in vision. Thus, anti-VEGF therapy, either alone or in conjunction with angiogenic therapies, is clearly a promising treatment for various forms of ocular neovascularization, including AMD and diabetic retinopathy.

Accordingly, the present invention features a method for treating a patient suffering from an ocular neovascular disease, which method includes the following steps: (a) administering to the patient an effective amount of an anti-VEGF aptamer; and (b) providing the patient with phototherapy, such as photodynamic therapy or thermal laser photocoagulation.

In one embodiment of the invention, the photodynamic therapy (PDT) includes the steps of: (i) delivering a photosensitizer to the eye tissue of a patient; and (ii) exposing the photosensitizer to light having a wavelength absorbed by the photosensitizer for a time and at an intensity sufficient to inhibit neovascularization in the patient's eye tissue. A variety of photosensitizers may be used, including but not limited to, benzoporphyrin derivatives (BPD), monoaspartyl chlorin e6, zinc phthalocyanine, tin etiopurpurin, tetrahydroxy tetraphenylporphyrin, and porfimer sodium (PHOTOFRIN®), and green porphyrins.

In a related aspect, the present invention provides a method for treating an ocular neovascular disease in a patient, which method involves administering to the patient: (a) an effective amount of an anti-VEGFaptamer; and (b) a second compound capable of diminishing or preventing the development of unwanted neovasculature. The anti-VEGF agents or other compounds that may be combined with anti-VEGF aptamers include, but are not limited to: antibodies or antibody fragments specific to VEGF; antibodies specific to VEGF receptors; compounds that inhibit, regulate, and/or modulate tyrosine kinase signal transduction; VEGF polypepides; oligonucleotides that inhibit VEGF expression at the nucleic acid level, for example antisense RNAs; retinoids; growth factor-containing compositions; antibodies that bind to collagens; and various organic compounds and other agents with angiogenesis inhibiting activity.

In a preferred embodiment of the invention, the anti-VEGF agent is a nucleic acid ligand to vascular endothelial growth factor (VEGF). The VEGF nucleic acid ligand may include ribonucleic acid, deoxyribonucleic acid, and/or modified nucleotides. In particularly preferred embodiments, the VEGF nucleic acid ligand includes 2′F-modified nucleotides, 2′-O-methyl (2′-OMe) modified nucleotides, and/or a polyalkylene glycol, such as polyethylene glycol (PEG). In some embodiments, the VEGF nucleic acid ligand is modified with a moiety, for example a phosphorothioate, that decreases the activity of endonucleases or exonucleases on the nucleic acid ligand relative to the unmodified nucleic acid ligand, without adversely affecting the binding affinity of the ligand.

In yet another aspect, the invention provides a method for treating an ocular neovascular disease in a patient, which method involves the steps of: (a) administering to the patient an effective amount of an agent that inhibits the development of ocular neovascularization, for example, an anti-VEGFaptamer; and (b) providing the patient with a therapy that destroys abnormal blood vessels in the eye, for example PDT.

The anti-VEGF aptamer may be administer intraocullary by injection into the eye. Alternatively, the aptamer may be delivered using an intraocular implant.

The methods of the invention can be used to treat a variety of neovascular diseases, including but not limited to, ischemic retinopathy, intraocular neovascularization, age-related macular degeneration, corneal neovascularization, retinal neovascularization, choroidal neovascularization, diabetic macular edema, diabetic retina ischemia, diabetic retinal edema, and proliferative diabetic retinopathy.

Other advantages and features of the present invention will be apparent from the following detailed description thereof and from the claims.

### Definitions

By “ocular neovascular disease” is meant a disease characterized by ocular neovascularization, i.e. the development of abnormal blood vessels in the eye of a patient.

By “patient” is meant any animal having ocular tissue that may be subject to neovascularization. Preferably, the animal is a mammal, which includes, but is not limited to, humans and other primates. The term also includes domesticated animals, such as cows, hogs, sheep, horses, dogs, and cats.

By “phototherapy” is meant any process or procedure in which a patient is exposed to a specific dose of light of a particular wavelength, including laser light, in order to treat a disease or other medical condition.

By “photodynamic therapy” or “PDT” is meant any form of phototherapy that uses a light-activated drug or compound, referred to herein as a photosensitizer, to treat a disease or other medical condition characterized by rapidly growing tissue, including the formation of abnormal blood vessels (i.e., angiogenesis). Typically, PDT is a two-step process that involves local or systemic administration of the photosensitizer to a patient followed by activation of the photosensitizer by irradiation with a specific dose of light of a particular wavelength.

By “anti-VEGF agent” is meant a compound that inhibits the activity or production of vascular endothelial growth factor (“VEGF”).

By “photosensitizer” or “photoactive agent” is meant a light-absorbing drug or other compound that upon exposure to light of a particular wavelength becomes activated thereby promoting a desired physiological event, e.g., the impairment or destruction of unwanted cells or tissue.

By “thermal laser photocoagulation” is meant a form of photo-therapy in which laser light rays are directed into the eye of a patient in order to cauterize abnormal blood vessels in the eye to seal them from further leakage.

By “effective amount” is meant an amount sufficient to treat a symptom of an ocular neovascular disease.

The term “light” as used herein includes all wavelengths of electromagnetic radiation, including visible light. Preferably, the radiation wavelength is selected to match the wavelength(s) that excite(s) the photosensitizer. Even more preferably, the radiation wavelength matches the excitation wavelength of the photosensitizer and has low absorption by non-target tissues.

### BRIEF DESCRIPTION OF THE DRAWING

FIG. 1 is the chemical structure of the anti-VEGF agent NX1838.

### DETAILED DESCRIPTION

VEGF (Vascular Endothelial Growth Factor) is an important stimulus for the growth of new blood vessels in the eye. We have discovered that anti-VEGF therapy provides a safe and effective treatment for neovascular disease, especially when combined with a secondary therapy that is able to reduce or eliminate ocular neovascularization, such as, for example, photodynamic therapy (PDT). We found that the combination of these therapies is far superior at treating conditions characterized by the development of unwanted neovasculature in the eye than most conventional treatments, including the use of either of these therapies alone.

Accordingly, the present invention provides a method of treating an ocular neovascular disease which involves administering to a patient an anti-VEGF agent and treating the patient with phototherapy (e.g., PDT) or with other therapies, such as photocoagulation, that destroy abnormal blood vessels in the eye. This method can be used to treat a number of ophthamalogical diseases and disorders marked by the development of ocular neovascularization, including but not limited to, ischemic retinopathy, intraocular neovascularization,. age-related macular degeneration, corneal neovascularization, retinal neovascularization, choroidal neovascularization, diabetic macular edema, diabetic retina ischemia, diabetic retinal edema, and proliferative diabetic retinopathy.

Anti-VEGF Therapy

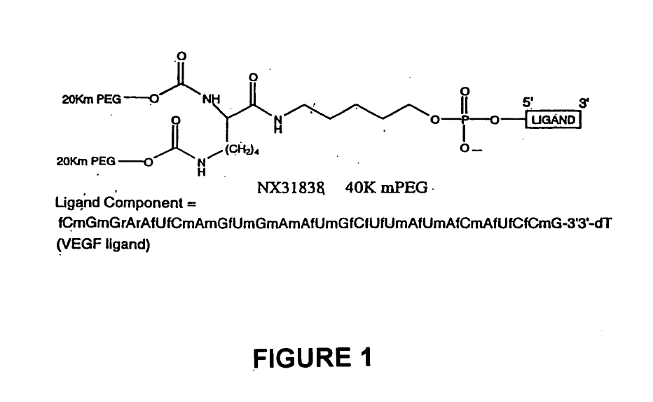

A variety of anti-VEGF therapies that inhibit the activity or production of VEGF, including aptamers and VEGF antibodies, are available and can be used in the methods of the present invention. The preferred anti-VEGF agents are nucleic acid ligands of VEGF, such as those described in U.S. Pat. Nos. 6,168,778 B1; 6,147,204; 6,051,698; 6,011,020; 5,958,691; 5,817,785; 5,811,533; 5,696,249; 5,683,867; 5,670,637; and 5,475,096. A particularly preferred anti-VEGF agent is EYE001 (previously referred to as NX1838), which is a modified, pegylated aptamer that binds with high affinity to the major soluble human VEGF isoform and has the general structure shown in FIG. 1 (described in U.S. Pat. No. 6,168,788; Journal of Biological Chemistry, Vol. 273(32): 20556-20567 (1998); and In Vitro Cell Dev. Biol.—Animal Vol. 35:533-542 (1999)).

Alternatively, the anti-VEGF agents may be, for example, VEGF antibodies or antibody fragments, such as those described in U.S. Pat. Nos. 6,100,07 1; 5,730,977; and WO 98/45331. Other suitable anti-VEGF agents or compounds that may be used in combination with anti-VEGF agents according to the present invention include, but are not limited to, antibodies specific to VEGF receptors (e.g., U.S. Pat. Nos. 5,955,311; 5,874,542; and 5,840,301); compounds that inhibit, regulate, and/or modulate tyrosine kinase signal transduction (e.g., U.S. Pat. No. 6,313,138 B1); VEGF polypepides (e.g., U.S. Pat. No. 6,270,933 B1 and WO 99/47677); oligonucleotides that inhibit VEGF expression at the nucleic acid level, for example antisense RNAs (e.g., U.S. Pat. Nos. 5,710,136; 5,661,135; 5,641,756; 5,639,872; and 5,639,736); retinoids (e.g., U.S. Pat. No. 6,001,885); growth factor-containing compositions (e.g., U.S. Pat. No. 5,919,459); antibodies that bind to collagens (e.g., WO 00/40597); and various organic compounds and other agents with angiogenesis inhibiting activity (U.S. Pat. Nos. 6,297,238 B1; 6,258,812 B1; and 6,114,320).

Administration of Anti-VEGF Agents

Once a patient has been diagnosed with a neovascular disorder of the eye, the patient is treated by administration of an anti-VEGF agent in order to block the negative effects of VEGF, thereby alleviating the symptoms associated with the neovascularization. As discussed above, a wide variety of anti-VEGF agents are known in the art and may be used in the present invention. Methods for preparing these anti-VEGF agents are also well-known and many are commercially available medications.

The anti-VEGF agents can be administered systemically, e.g. orally or by IM or IV injection, in admixture with a pharmaceutically acceptable carrier adapted for the route of administration. A variety of physiologically acceptable carriers can be used to administer the anti-VEGF agents and their formulations are known to those skilled in the art and are described, for example, in Remington's Pharmaceutical Sciences, (18^{th} edition), ed. A. Gennaro, 1990, Mack Publishing Company, Easton, Pa. and Pollock et al.

The anti-VEGF agents are preferably administered parenterally (e.g., by intramuscular, intraperitoneal, intravenous, intraocular, intravitreal, or subcutaneous injection or implant). Formulations for parenteral administration include sterile aqueous or non-aqueous solutions, suspensions, or emulsions. A variety of aqueous carriers can be used, e.g., water, buffered water, saline, and the like. Examples of other suitable vehicles include polypropylene glycol, polyethylene glycol, vegetable oils, gelatin, hydrogenated naphalenes, and injectable organic esters, such as ethyl oleate. Such formulations may also contain auxiliary substances, such as preserving, wetting, buffering, emulsifying, and/or dispersing agents. Biocompatible, biodegradable lactide polymer, lactide/glycolide copolymer, or polyoxyethylene-polyoxypropylene copolymers may be used to control the release of the active ingredients.

Alternatively, the anti-VEGF agents can be administered by oral ingestion. Compositions intended for oral use can be prepared in solid or liquid forms, according to any method known to the art for the manufacture of pharmaceutical compositions. The compositions may optionally contain sweetening, flavoring, coloring, perfuming, and preserving agents in order to provide a more palatable preparation.

Solid dosage forms for oral administration include capsules, tablets, pills, powders, and granules. Generally, these pharmaceutical preparations contain active ingredient admixed with non-toxic pharmaceutically acceptable excipients. These may include, for example, inert diluents, such as calcium carbonate, sodium carbonate, lactose, sucrose, glucose, mannitol, cellulose, starch, calcium phosphate, sodium phosphate, kaolin and the like. Binding agents, buffering agents, and/or lubricating agents (e.g., magnesium stearate) may also be used. Tablets and pills can additionally be prepared with enteric coatings.

Liquid dosage forms for oral administration include pharmaceutically acceptable emulsions, solutions, suspensions, syrups, and soft gelatin capsules. These forms contain inert diluents commonly used in the art, such as water or an oil medium, and can also include adjuvants, such as wetting agents, emulsifying agents, and suspending agents.

The anti-VEGF agents can also be administered topically, for example, by patch or by direct application to the eye, or by iontophoresis.

The anti-VEGF agents may be provided in sustained release compositions, such as those described in, for example, U.S. Pat. Nos. 5,672,659 and 5,595,760. The use of immediate or sustained release compositions depends on the nature of the condition being treated. If the condition consists of an acute or over-acute disorder, treatment with an immediate release form will be preferred over a prolonged release composition. Alternatively, for certain preventative or long-term treatments, a sustained released composition may be appropriate.

The anti-VEGF agent may also be delivered using an intraocular implant. Such implants may be biodegradable and/or biocompatible implants, or may be non-biodegradable implants. The implants may be permeable or impermeable to the active agent, and may be inserted into a chamber of the eye, such as the anterior or posterior chambers or may be implanted in the schelra, transchoroidal space, or an avascularized region exterior to the vitreous. In a preferred embodiment, the implant may be positioned over an avascular region, such as on the sclera, so as to allow for transcleral diffusion of the drug to the desired site of treatment, e.g. the intraocular space and macula of the eye. Furthermore, the site of transcleral diffusion is preferably in proximity to the macula.

Examples of implants for delivery of an anti-VEGF agent include, but are not limited to, the devices described in U.S. Pat. Nos. 3,416,530; 3,828,777; 4,014,335; 4,300,557; 4,327,725; 4,853,224; 4,946,450; 4,997,652; 5,147,647; 5,164,188; 5,178,635; 5,300,114; 5,322,691; 5,403,901; 5,443,505; 5,466,466; 5,476,511; 5,516,522; 5,632,984; 5,679,666; 5,710,165; 5,725,493; 5,743,274; 5,766,242; 5,766,619; 5,770,592; 5,773,019; 5,824,072; 5,824,073; 5,830,173; 5,836,935; 5,869,079, 5,902,598; 5,904,144; 5,916,584; 6,001,386; 6,074,661; 6,110,485; 6,126,687; 6,146,366; 6,251,090; and 6,299,895, and in WO 01/30323 and WO 01/28474, all of which are incorporated herein by reference.

Dosage

The amount of active ingredient that is combined with the carrier materials to produce a single dosage will vary depending upon the subject being treated and the particular mode of administration. Generally, the anti-VEGF agent should be administered in an amount sufficient to reduce or eliminate a symptom of an ocular neovascular disease.

Dosage levels on the order of about 1 μg/kg to 100 mg/kg of body weight per administration are useful in the treatment of the above mentioned neovascular disorders. When administered directly to the eye, the preferred dosage range is about 0.3 mg to about 3 mg per eye. The dosage may be administered as a single dose or divided into multiple doses. In general, the desired dosage should be administered at set intervals for a prolonged period, usually at least over several weeks, although longer periods of administration of several months or more may be needed.

One skilled in the art will appreciate that the exact individual dosages may be adjusted somewhat depending on a variety of factors, including the specific anti-VEGF agent being administered, the time of administration, the route of administration, the nature of the formulation, the rate of excretion, the particular disorder being treated, the severity of the disorder, and the age, weight, health, and gender of the patient. Wide variations in the needed dosage are to be expected in view of the differing efficiencies of the various routes of administration. For instance, oral administration generally would be expected to require higher dosage levels than administration by intravenous or intravitreal injection. Variations in these dosage levels can be adjusted using standard empirical routines for optimization, which are well-known in the art. The precise therapeutically effective dosage levels and patterns are preferably determined by the attending physician in consideration of the above identified factors.

In addition to treating pre-existing neovascular diseases, anti-VEGF agents can be administered prophylactically in order to prevent or slow the onset of these disorders. In prophylactic applications, an anti-VEGF agent is administered to a patient susceptible to or otherwise at risk of a particular neovascular disorder. Again, the precise amounts that are administered depend on various factors such as the patient's state of health, weight, etc.

Effectiveness of Anti-VEGF Therapy

In order to assess the effectiveness of anti-VEGF therapy to treat ocular neovascularization, we conducted a number of studies, which are described in the examples below, that involved the administration of an anti-VEGF aptamer with and without photodynamic therapy in patients suffering from subfoveal choroidal neovascularization secondary to age-related macular degeneration. A Phase 1A single intravitreal injection study of anti-VEGF therapy for patients with subfoveal choroidal neovascularization (CNV) secondary to age-related macular degeneration (AMD) revealed an excellent safety profile (Example 6). Ophthalmic evaluation revealed that 80% of patients showed stable or improved vision 3 months after treatment and that 27% of eyes demonstrated a 3-line or greater improvement in vision on the ETDRS chart at this time period. No significant related adverse events were reported locally or systemically. These data demonstrated that anti-VEGF therapy is a promising new avenue for the treatment of neovascular diseases of the eye, including exudative macular degeneration and diabetic retinopathy.

We also performed a Phase 1B multiple descending dose safety study of anti-VEGF therapy using multiple intravitreal injections of the anti-VEGF aptamer with or without photodynamic therapy in patients with subfoveal CNV secondary to AMD (Example 7). The safety study showed no significant safety issues related to the drug. Ophthalmic evaluation revealed that 87.5% of patients that received the anti-VEGF aptamer alone showed stable or improved vision 3 months after treatment and that 25% of eyes demonstrated a 3-line or greater improvement in vision on the ETDRS chart at this time period. A 60% 3-line gain at 3 months was noted in patients that received both the anti-VEGF aptamer and photodynamic therapy. Multiple intravitreal injections of the anti-VEGF aptamer were very well tolerated in this Phase 1B study.

The results of this Phase 1B multiple intravitreal injection clinical study of anti-VEGF therapy (Example 7) expand the excellent safety profile reported by our Phase 1A single-injection study (Example 6). Specifically, the Phase 1B study shows the intraocular and systemic safety of three consecutive anti-VEGF aptamer intravitreal injections given monthly. No serious related adverse events were noted. The adverse events encountered appeared to be unrelated or minor events in some cases probably due to the intravitreal injection itself.

The 3-line gain observed in 25% of the aptamer only treated group at 3 months compares favorably to historical controls such as the results of the pivotal trial of PDT (2.2%) and its controls (1.4%) at 3 months (Arch Ophthalmol 1999, 117:1329-1345) and a sham radiation control group (3%) (Ophthalmology 1999, 106;12:2239-2247) where no more than 3% of patients showed such an improvement at this same time period.

The 25% 3-line gain at 3 months is consistent with the 26.7% improvement rate noted in the Phase 1A study of the aptamer. It may be that the anti-permeability effects of the drug caused resorption of subretinal fluid and, thus improved vision in these cases. Interestingly, a recent study using an anti-VEGF antibody fragment from Genentech also showed a 26% 3-line gain rate in a Phase 1 clinical trial. This antibody fragment shares the same mechanism of blocking extracellular VEGF as the anti-VEGF aptamer.

The stabilization or improvement rate of 87.5% observed at 3 months in the Phase IB study also compares favorably with the 50.5% rate for the PDT-treated patients in that pivotal trial (Arch Ophthalmol 1999, 117:1329-1345), the 44% rate in the PDT controls, and 48% rate in the sham radiation control group (Ophthalmology 1999, 106; 12:2239-2247).

The 60% 3-line gain at 3 months in the patients that received both the anti-VEGF aptamer and PDT was also very encouraging. In the pivotal Phase 3 PDT trial only 2.2% of patients showed such visual improvement (Arch Ophthalmol 1999, 117:1329-1345). Both of these study groups included eyes with classic subfoveal CNV. The improvement in vision observed in these eyes is supported by the finding that the investigators choose to re-treat with PDT at 3 months in only 40% of cases compared to the 93% re-treatment rate reported in the pivotal PDT trial (Arch Ophthalmol 1999, 117:1329-1345).

In addition, numerous pre-clinical studies now show that anti-VEGF therapy can prevent VEGF-induced neovascularization of the cornea, iris, retina, and choroid (Arch Ophthalmol 1996, 114:66-7; Invest Ophthalmol Vis Sci 1994, 35:101). The pre-clinical studies described below in Examples 1-5 with EYE001 provide evidence that anti-VEGF therapy may be useful in decreasing vascular permeability and ocular neovascularization. The anti-VEGF aptamer showed great efficacy in the ROP retinal neovascularization model where 80% of retinal neovascularization was inhibited compared to controls (p=0.0001). The Miles assay model showed almost complete attenuation of VEGF mediated vascular leakage following addition of EYE001 and the corneal angiogenesis model also showed a significant reduction in neovascularization with EYE001. The Miles Assay study in guinea pigs suggests that the anti-VEGF aptamer can significantly decrease vascular permeability. This property of decreasing vascular permeability may prove to be clinically important for decreasing fluid and edema in CNV and diabetic macular edema. Thus, anti-VEGF therapy may act both as an anti-permeability and/or anti-angiogenic agent.

Photodynamic Therapy (PDT)

As discussed above, one embodiment of the method of the invention involves administering an anti-VEGF agent in combination with photodynamic therapy (PDT). PDT is a two-step process that starts with the local or systemic administration of a light-absorbing photosensitive agent, such as a porphyrin derivative, that accumulates selectively in target tissues of the patient. Upon irradiation with light of an activating wavelength, reactive oxygen species are produced in cells containing the photosensitizer, which promote cell death. For example, in the treatment of eye diseases characterized by ocular neovascularization, a photosensitizer is selected that accumulates in the neovasculature of the eye. The patient's eye is then exposed to light of an appropriate wavelength, which results in the destruction of the abnormal blood vessels, thereby improving the patient's visual acuity.

Photosensitizers

The photodynamic therapy according to the invention can be performed using any of a number of photoactive compounds. For example, the photosensitizer can be any chemical compound that collects in one or more types of selected target tissues and, when exposed to light of a particular wavelength, absorbs the light and induces impairment or destruction of the target tissues. Virtually any chemical compound that homes to a selected target and absorbs light may be used in this invention. Preferably, the photosensitizer is nontoxic to the animal to which it is administered and is capable of being formulated in a nontoxic composition. The photosensitizer is also preferably nontoxic in its photodegraded form. Ideal photosensitizers are characterized by a lack of toxicity to cells in the absence of the photochemical effect and are readily cleared from non-target tissues.

A comprehensive listing of photosensitizers may be found, for example, in Kreimer-Birnbaum, Sem. Hematol. 26:157-73, 1989. Photosensitive compounds include, but are not limited to, chlorins, bacteriochlorins, phthalocyanines, porphyrins, purpurins, merocyanines, pheophorbides, psoralens, aminolevulinic acid (ALA), hematoporphyrin derivatives, porphycenes, porphacyanine, expanded porphyrin-like compounds and pro-drugs such as δ-aminolevulinic acid, which can produce drugs such as protoporphyrin. (See, e.g., photosenitizers described in any of U.S. Pat. Nos. 5,438,071; 5,405,957; 5,198,460; 5,190,966; 5,173,504; 5,171,741; 5,166,197; 5,095,030; 5,093,349; 5,079,262; 5,028,621; 5,002,962; 4,968,715; 4,920,143; 4,883,790; 4,866,168; and 4,649,151.) Preferred photosensitizing agents are benzoporphyrin derivatives (BPD), monoaspartyl chlorin e6, zinc phthalocyanine, tin etiopurpurin, tetrahydroxy tetraphenylporphyrin, and porfimer sodium (PHOTOFRIN®). A particularly potent group of photosensitizers includes green porphyrins, which are described in detail in Levy el al., U.S. Pat. No. 5,171,749.

Any of the photosensitizers described above can be used in the methods of the invention. Of course, mixtures of two or more photoactive compounds can also be used; however, the effectiveness of the treatment depends on the absorption of light by the photosensitizer so that if mixtures are used, components with similar absorption maxima are preferred.

The photosensitizing agents of the present invention preferably have an absorption spectrum that is within the range of wavelengths between 350 nm and 1200 nm, preferably between about 400 and 900 nm and, most preferably, between 600 and 800 nm.

The photosensitizer is formulated so as to provide an effective concentration to the target ocular tissue. The photosensitizer may be coupled to a specific binding ligand which may bind to a specific surface component of the target ocular tissue or, if desired, by formulation with a carrier that delivers higher concentrations to the target tissue. The nature of the formulation will depend in part on the mode of administration and on the nature of the photosensitizer selected. Any pharmaceutically acceptable excipient, or combination thereof, appropriate to the particular photoactive compound may be used. Thus, the photosensitizer may be administered as an aqueous composition, as a transmucosal or transdermal composition, or in an oral formulation.

As previously mentioned, the method of the invention is particularly effective to treat patients suffering from loss of visual acuity associated with unwanted neovasculature. Increased numbers of LDL receptors have been shown to be associated with neovascularization. Green porphyrins, and in particular BPD-MA, strongly interact with such lipoproteins. LDL itself can be used as a carrier for green porphyrins, or liposomal formulations may be used. Liposomal formulations are believed to deliver green porphyrins selectively to the low-density lipoprotein component of plasma which, in turn acts as a carrier to deliver the active ingredient more effectively to the desired site. By increasing the partitioning of the green porphyrin into the lipoprotein phase of the blood, liposomal formulations can result in a more efficient delivery of the photosensitizer to neovasculature. Compositions of green porphyrins involving lipocomplexes, including liposomes, are described in U.S. Pat. No. 5,214,036. Liposomal BPD-MA for intravenous administration can be obtained from QLT PhotoTherapeutics Inc., Vancouver, British Columbia.

The photosensitizer can be administered locally or systemically in any of a wide variety of ways, for example, orally, parenterally (e.g., intravenous, intramuscular, intraperitoneal or subcutaneous injection), topically via patches or implants, or the compound may be placed directly in the eye. The photosensitizing agent can be administered in a dry formulation, such as pills, capsules, suppositories, or patches. The photosensitizing agent also may be administered in a liquid formulation, either alone with water, or with pharmaceutically acceptable excipients, such as are disclosed in Remington's Pharmaceutical Sciences, supra. The liquid formulation also can be a suspension or an emulsion. Suitable excipients for suspensions for emulsions include water, saline, dextrose, glycerol, and the like. These compositions may contain minor amounts of nontoxic auxiliary substances such as wetting or emulsifying agents, antioxidants, pH buffering agents, and the like.

The dose of photosensitizer can vary widely depending a variety of factors, such as the type of photosensitizer; the mode of administration; the formulation in which it is carried, such as in the form of liposomes; or whether it is coupled to a target-specific ligand, such as an antibody or an immunologically active fragment. Other factors which impact the dose of photosensitizing agent include the target cell(s) sought, the patient's weight, and the timing of the light treatment. While various photoactive compounds require different dosage ranges, if green porphyrins are used, a typical dosage is of the range of 0.1-50 mg/M² (of body surface area) preferably from about 1-10 mg/M² and even more preferably about 2-8 mg/M².

The various parameters used for photodynamic therapy in the invention are interrelated. Therefore, the dose should also be adjusted with respect to other parameters, for example, fluence, irradiance, duration of the light used in photodynamic therapy, and time interval between administration of the dose and the therapeutic irradiation. All of these parameters should be adjusted to produce significant enhancement of visual acuity without significant damage to the eye tissue.

Light Treatment

After the photosensitizer has been administered to the patient, the target ocular tissue is irradiated with light at a wavelength that is absorbed by the photosensitizer that was used. The spectra for the photosensitizers described herein are known in the art; for any particular photoactive compound, it is a trivial matter to ascertain the spectrum. For green porphyrins, the desired wavelength range is generally between about 550 and 695 nm. A wavelength in this range is especially preferred for enhanced penetration into bodily tissues.

As a result of being exposed to light, the photosensitizer enters an excited state and is believed to interact with other compounds to form reactive intermediates, such as singlet oxygen, which can cause disruption of cellular structures. Possible cellular targets include the cell membrane, mitochondria, lysosomal membranes, and the nucleus. Evidence from tumor and neovascular models indicates that occlusion of the vasculature is a major mechanism of photodynamic therapy, which occurs by damage to endothelial cells, with subsequent platelet adhesion, degranulation, and thrombus formation.

The fluence during the irradiating treatment can vary widely, depending on type of tissue, depth of target tissue, and the amount of overlying fluid or blood, but preferably varies from about 50-200 Joules/cm².

The irradiance typically varies from about 150-900 mW/cm², with the range between about 150-600 mW/cm² being preferred. However, the use of higher irradiances may be selected as effective and having the advantage of shortening treatment times.

The optimum time following photoactive agent administration until light treatment can also vary widely depending on the mode of administration, the form of administration, and the specific ocular tissue being targeted. Typical times after administration of the photoactive agent range from about 1 minute to about 2 hours, preferably about 5-30 minutes, and more preferably about 10-25 minutes.

The duration of radiation exposure is preferably between about 1 and 30 minutes, depending on the power of the radiation source. The duration of light irradiation also depends on the fluence desired. For example, for an irradiance of 600 mW/cm², a fluence of 50 J/cm² requires 90 seconds of irradiation; 150 J/cm² requires 270 seconds of irradiation.

The radiation is further defined by its intensity, duration, and timing with respect to dosing with the photosensitive agent (post injection interval). The intensity must be sufficient for the radiation to penetrate skin and/or to reach the target tissues to be treated. The duration must be sufficient to photoactivate enough photosensitive agent to act on the target tissues. Both intensity and duration must be limited to avoid overtreating the patient. The post injection interval before light application is important, because in general the sooner light is applied after the photosensitive agent is administered, 1) the lower is the required amount of light and 2) the lower is the effective amount of photosensitive agent.

Clinical examination and fundus photography typically reveal no color change immediately following photodynamic therapy, although a mild retinal whitening occurs in some cases after about 24 hours. Closure of choroidal neovascularization is preferably confirmed histologically by the observation of damage to endothelial cells. Observations to detect vacuolated cytoplasm and abnormal nuclei associated with disruption of neovascular tissue may also be evaluated.

In general, effects of the photodynamic therapy as regards reduction of neovascularization can be performed using standard fluorescein angiographic techniques at specified periods after treatment. The effectiveness of PDT may also be determined through a clinical evaluation of visual acuity, using means standard in the art, such as conventional eye charts in which visual acuity is evaluated by the ability to discern letters of a certain size, usually with five letters on a line of given size.

Other Therapies for Treating Neovascular Disease

In addition to PDT, there are a number of other therapies for treating neovascular disease which may be used in combination with anti-VEGF therapies. For example, a form of photo-therapy known as Thermal Laser Photocoagulation is a standard ophthalmic procedure for the treatment of a range of eye disorders, including retinal vascular problems (e.g. diabetic retinopathy), choroidal vascular problems and macular lesions (e.g. senile macular degeneration). This procedure involves the use of laser light to cauterize abnormal blood vessels in the eye of a patient in order to seal them from further leakage. (See, e.g. Arch. Ophthalmol. 1991, 109:1109-1114). Alternatively, compounds capable of diminishing or preventing the development of unwanted neovasculature, including other anti-VEGF agents, anti-angiogenesis agents, or other agents that inhibit the development of ocular neovascularization may be used in combination with anti-VEGF therapy.

The features and other details of the invention will now be more particularly described and pointed out in the following examples describing preferred techniques and experimental results. These examples are provided for the purpose of illustrating the invention and should not be construed as limiting.

### EXAMPLES

In the following Examples, the anti-VEGF pegylated aptamer EYE001 was used. As discussed above, this aptamer is a polyethylene glycol (PEG)-conjugated oligonucleotide that binds to the major soluble human VEGF isoform, VEGF₁₆₅, with high specificity and affinity. The aptamer binds and inactivates VEGF in a manner similar to that of a high-affinity antibody directed towards VEGF. Examples 1-5 report the pre-clinical results of studies with the anti-VEGF aptamer in various models of ocular neovascularization, Example 6 reports the clinical phase IA safety results in humans with exudative AND, and Example 7 reports the clinical phase IB results. Generally, dosages and concentrations are expressed as the oligonucleotide weight of EYE001 (NX1838) only and are based on an approximate extinction coefficient for the aptamer of 37 μg/mL/A₂₆₀ unit.

### Example 1

Cutaneous Vascular Permeability Assay (Miles Assay)

One of the biological activities of VEGF is to increase vascular permeability through specific binding to receptors on vascular endothelial cells. The interaction results in relaxation of the tight endothelial junctions with subsequent leakage of vascular fluid. Vascular leakage induced by VEGF can be measured in-vivo by following the leakage of Evans Blue Dye from the vasculature of the guinea pig as a consequence of an intradermal injection of VEGF (Dvorak H F, Brown L F, Detmar M, Dvorak A M. Vascular Permeability Factor/Vascular Endothelial Growth Factor, Microvascular Hyperpermeability, and Angiogenesis. Am J Pathol. 1995, 146:1029.) Similarly, the assay can be used to measure the ability of a compound to block this biological activity of VEGF.

VEGF₁₆₅ (20-30 nM) was premixed ex-vivo with EYE001 (30 nM to 1 μM) and subsequently administered by intradermal injection into the shaved skin on the dorsum of guinea pigs. Thirty minutes following injection, the Evans Blue dye leakage around the injection sites was quantified by use of a computerized morphometric analysis system. The data (not shown) demonstrated that VEGF-induced leakage of the indicator dye from the vasculature can be almost completely inhibited by the co-administration of EYE001 at concentrations as low as 100 nM.

### Example 2

Corneal Angiogenesis Assay

Methacyrate polymer pellets containing VEGF₁₆₅ (3 pmol) were implanted into the corneal stroma of rats to induce blood vessel growth into the normally avascular cornea. EYE001 was administered intravenously to the rats at doses of 1, 3, and 10 mg/kg either once or twice daily for 5 days. At the end of the treatment period, all of the individual corneas were photomicrographed. The extent to which new blood vessels developed in the corneal tissue, and their inhibition by EYE001, were quantified by standardized morphometric analysis of the photomicrographs.

The data (not shown) demonstrated that systemic treatment with EYE001 results in significant inhibition (65%) of VEGF-dependent angiogenesis in the cornea when compared to treatment with phosphate buffered saline (PBS). Once daily treatment with 10 mg/kg was as effective as twice daily treatment. The 3 mg/kg dose had activity similar to the 10 mg/kg dose but significant efficacy was not evident at 1 mg/kg.

### Example 3

Retinopathy of Prematurity Study

Even though ROP is clearly distinct from diabetic retinopathy and AMD, the mouse model of ROP has been used to demonstrate a role for VEGF in the abnormal retinal vascularization that occurs in this disease (Smith L E, Wesolowski E, McLellan A, Kostyk S K, Amato D R, Sullivan R, D'Amore P A. Oxygen-induced retinopathy in the mouse. Invest Ophthalmol Vis Sci. 1994,35:101.) These data provided a rationale for studying the anti-angiogenic properties of EYE001 in this model.

Litters of 9, 8, 8, 7 and 7 mice, respectively, were left in room air or made hyperoxic and were treated intraperitoneally with PBS or EYE001 (1, 3, or 10 mg/kg/day). The endpoint of the assay, outgrowth of new capillaries through the inner limiting membrane of the retina into the vitreous humor, was assessed by microscopic identification and counting of the neovascular buds in 20 histologic sections of each eye from all of the treated and control mice. A reduction in retinal neovasculature of 80% relative to the untreated control was seen at both the 10 mg/kg and 3 mg/kg doses (p=0.0001 for both).

### Example 4

Human Tumor Xenografts

The in-vivo efficacy of EYE001 was tested in human tumor xenografts (A673 rhabdomyosarcoma and Wilms tumor) implanted in nude mice. In both cases, mice were treated with 10 mg/kg EYE001 given intraperitoneally once a day following development of established tumors (200 mg). Control groups were treated with a sequence scrambled control aptamer (oligonucleotide).

Treatment of mice with 10 mg/kg of EYE001 once daily inhibited A673 rhabdomyosarcoma tumor growth by 80% and Wilms tumor by 84% relative to the control. In the Wilms tumor model, two weeks after termination of therapy, tumor size rebounded so vigorously in treated animals that there was no longer any difference in tumor size compared to controls.

### Example 5

Intravitreal Pharmacokinetics of EYE001 in Rabbits

Rabbits were obtained and cared for in accordance with all applicable state and federal guidelines and adhered to the “Principles of Laboratory Animal Care” (NIH publication #85-23, revised 1985). A total of 18 male New Zealand White rabbits were administered EYE001 by intravitreous injection. Each animal received a dose as a bilateral injection of 0.50 mg/eye (1.0 mg/animal) in a volume of 40 μL/eye. EDTA-Plasma and vitreous humor samples were collected over a 28-day period following dose administration and stored frozen (−70° C.) until assayed. Vitreous humor from each eye was collected separately after the animals were sacrificed by exsanguination. EYE001 concentrations in vitreous humor samples were determined by an HPLC assay method similar to that described previously by Tucker et al. (Detection and plasma pharmacokinetics of an anti-vascular endothelial growth factor oligonucleotide-aptamer (NX1838) in rhesus monkeys. J. Chromatogr. Biomed. Appl. 1999, 732:203-212) and by a dual hybridization assay method similar to that described previously by Drolet et al. (Pharmacokinetics and Safety of an Anti-Vascular Endothelial Growth Factor Aptamer (NX1838) Following Injection into the Vitreous Humor of Rhesus Monkeys. Pharm. Res., 2000, 17:1503-1510.) Thevitreoushumorconcentration was calculated by averaging the results from both assays. EYE001 concentrations in plasma were determined only by the dual hybridization assay.

Following a single dose of EYE001 as a bilateral administration of 0.50 mg/eye (1.0 mg/animal), the initial vitreous humor levels were approximately 350 μg/mL and decreased by an apparent first order elimination process to approximately 1.7 μg/mL by day 28. The estimated terminal half-life was 83 hours similar to the 94-hour half-life observed in rhesus monkeys (Drolet et al., supra). At four weeks following administration of EYE001, drug levels in the vitreous humor (˜190 nM) remained well above the KD for VEGF (200 pM) suggesting that once monthly dosing in humans is appropriate, assuming that pharmacokinetic parameters are comparable in the rabbit and human vitreous humor. In contrast to the high levels of EYE001 found in the vitreous humor, the plasma concentrations were significantly lower and ranged from 0.092 to 0.005 μg/mL from day 1 to day 21. Plasma levels declined by an apparent first order elimination process as well with an estimated terminal half-life of 84 hours. The plasma terminal half-life thus mimicked the vitreous humor half-life as observed in rhesus monkeys (Drolet et al., supra) and is indicative of classical flip-flop kinetics in which the clearance from the eye is the rate-determining step for plasma clearance. These data are consistent with a highly stable (nuclease resistant) aptamer that undergoes a slow rate of release from the vitreous humor into the systemic circulation.

### Example 6

Clinical Trial-Phase IA Study

We performed a multi-centered, open-label, dose-escalation study of a single intravitreous injection of EYE001 in patients with subfoveal CNV secondary to age-related macular degeneration and with a visual acuity worse than 20/200 on the ETDRS chart. The starting dose was 0.25 mg injected once intravitreously. Dosages of 0.5, 1, 2 and 3 mg were also tested. Complete ophthalmic examination with fundus photography and fluorescein angiography was performed. A total of 15 patients were treated.

Selection Criteria.

Patients for the study were selected using the following inclusion and exclusion criteria:

Inclusion Criteria: Patients were required to be >50 years and in generally good health, have a best corrected visual acuity in the study eye worse than 20/200 on the ETDRS chart, and 20/400 or worse for at least the first patient of each cohort (n=3); best corrected visual acuity in the fellow eye equal to or better than 20/64; subfoveal CNV (classic and/or occult CNV) of >3.5 Macular Photocoagulation Study (MPS) disc areas in size; clear ocular media and adequate pupillary dilatation to permit good quality stereoscopic fundus photography; and intraocular pressure of 22 mmHg or less.

Exclusion Criteria: Exclusions included significant media opacities, including cataract, which might interfere with visual acuity, assessment of toxicity, or fundus photography, presence of ocular disease, including glaucoma, diabetic retinopathy, retinal vascular occlusion or other conditions (other than CNV from AMD) which might significantly affect vision; presence of other causes of CNV, including pathologic myopia (spherical equivalent of −8 diopters or more negative), the ocular histoplasmosis syndrome, angioid streaks, choroidal rupture and multifocal choroiditis; patients in whom additional laser treatment for CNV might be indicated or considered; any intraocular surgery within 3 months of study entry; blood occupying >50% of the lesion; previous vitrectomy; previous or concomitant therapy with another investigational agent to treat AMD except multivitamins and trace minerals; any of the following underlying systemic diseases including uncontrolled diabetes mellitus or presence of diabetic retinopathy; cardiac disease including myocardial infarction within 12 months prior to study entry, and/or coronary disease associated with clinical symptoms, and/or indications of ischemia noted on ECG; stroke (within 12 months of study entry); active bleeding disorders; any major surgical procedure within one month of study entry; active peptic ulcer disease with bleeding within 6 months of study entry; and concomitant systemic therapy with corticosteroids (e.g. oral prednisone), or other anti-angiogenic drugs (e.g. thalidomide).

Study Medication.

The drug product was a ready-to-use sterile solution composed of EYE001 (formerly NX1838) dissolved in 10 mM sodium phosphate and 0.9% sodium chloride buffer injection and presented in a sterile and pyrogen free 1 cc glass body syringe barrel, with a coated stopper attached to a plastic plunger, and a rubber end cap on the pre-attached 27 gauge needle. The pegylated aptamer was supplied at active drug concentrations of 1, 2.5, 5, 10, 20 or 30 mg/ml of EYE001 (expressed as oligonucleotide content) in order to provide a 100 μl delivery volume.

Patient Enrollment.

Before recruitment of patients into the study, written Institutional Review Board (IRB) approval of the protocol, informed consent and any additional patient information was obtained.

Results.

A single dose-ranging safety study was performed in 15 patients at doses varying from 0.25 to 3.0 mg/eye without reaching dose-limiting toxicity. Viscosity of the formulation prevented further dose escalation past 3 mg. Patients ranged in age from 64 to 92 years old. Eight males and seven females were entered and all were Caucasian. Eleven of the fifteen patients experienced a total of seventeen mild or moderate, adverse events including six, which were probably or possibly related to administration of EYE001: mild intraocular inflammation, scotoma, visual distortion, hives, eye pain and fatigue. In addition, there was one severe adverse event, which was unrelated to test drug. This was the diagnosis of breast carcinoma in one patient, where the lump had been noted prior to treatment.

At 3 months after injection of EYE001, 12 out of 15 (80%) eyes showed stable or improved vision. Four patients (26.7%) had significantly improved vision at the same time point, which was defined as a 3-line, or greater, increase in vision on the ETDRS chart. Patients with such improved vision at 3 months noted increases of +6, +4 and +3 lines on an ETDRS chart. No unexpected visual safety events were noted. Evaluation of color photographs and fluorescein angiograms revealed no signs of retinal or choroidal toxicity.

Our Phase IA clinical study showed that single intravitreal doses of the anti-VEGF aptamer could be administered safely up to 3 mg/eye. No significant ocular or systemic side effects were noted.

Clinicians agree that a minimum of one-year follow-up is desirable to evaluate any potential treatment for exudative AMD. Nevertheless, 3-month data is available from some prospective studies and is useful to assess both ophthalmic safety and any potential trends of a new therapy.

Historical controls indicate that only 1.4% (pivotal photodynamic trial) (Arch Ophthalmol 1999, 117:1329-1345) and 3.0% (radiation study) (1999, 106; 12:2239-2247) of eyes have shown significant visual improvement as defined by a gain of 3 or more lines on an ETDRS chart at 3 months. In addition, the PDT-treated group of the TAP study (Arch Ophthalmol 1999, 117:1329-1345) only noted such improved vision in 2.2% of cases at 3 months. These findings confirm our clinical impression that it is rare to see significant visual improvement at any time frame with any type (classic, occult or mixed) of CNV secondary to AMD.

In our study, at three months after intravitreal administration of the anti-VEGF aptamer, 80% of eyes showed stabilized or improved vision with 26.7% showing an increase in 3 or more lines on the ETDRS chart. These visual improvements are supported by clinical and angiographic findings in some of the aptamer-treated patients. Stabilization of vision has always been the goal of exudative AMD studies, so the significant visual acuity improvement (3 ETDRS lines) seen in 26.7% of patients at 3 months with only one dose was unexpected. Clearly, historical controls are inappropriate for comparison. In addition, the short follow-up period, small sample size, and different CNV type (i.e. percentage of classic, occult, or mixed CNV) precluded any final conclusions or comparisons. However, it appears that the aptamer-treated eyes have certainly shown at least excellent visual safety at 3 months and justify further studies.

In summary, pre-clinical and early clinical results with single intravitreal injections of the anti-VEGF aptamer are very encouraging. The safety of single-dose intravitreal injections of dosages up to 3 mg/eye has been established.

### Example 7

Clinical Trial-Phase IB Study

We conducted a multi-center, open-label, repeat dose Phase IB study of 3 mg/eye of EYE001 (anti-VEGF aptamer) in patients with subfoveal CNV secondary to AMD with a visual acuity worse than 20/100 in the study eye and better or equal to 20/400 in the fellow eye. If 3 or more patients experienced Dose-Limiting Toxicity (DLT's), the dose was reduced to 2 mg and then 1 mg, if necessary. The intended number of patients to be treated was 20; 10 patients with the anti-VEGF aptamer alone and 10 patients with both anti-VEGF therapy and PDT. Eleven sites in the U.S. were selected for the studies.

Definition of DLT(s)

If a patient in the study experienced any of the following DLTs, the dosage was reduced as described above:

Ophthalmic DLT:

Photographic Evaluation.

Accelerated formation of cataract: progression of one unit defined by the Age-Related Eye Disease Study (AREDS) Lens Opacity Grading Protocol as adapted from the Wisconsin Cataract Grading System.

Clinical Examination.

Clinically significant inflammation, which was severe (obscuring visualization of the retinal vasculature) and vision threatening.

Other ocular abnormalities not usually seen in patients with AMD, such as retinal, arterial, or venous occlusion, acute retinal detachment, and diffuse retinal hemorrhage.

Visual acuity: doubling or worsening of the visual angle (loss of ≧15 letters); transition to no light perception (NLP) for patients whose beginning visual acuity score is less than 15 letters unless the loss of vision is due to a vitreous hemorrhage related to the injection procedure between Days 2 through 7, Days 30-35, or Days 58-63.

Tonometry: increase from baseline of intraocular pressure (IOP) by ≧25 mmHg on two separate examinations at least one day apart or a sustained pressure of 30 mmHg for more than a week despite pharmacological intervention.

Fluorescein Angiogram

Significant retinal or choroidal vascular abnormalities not seen at baseline, such as: choroidal nonperfusion (effecting one or more quadrants) delay in arterio-venous transit times (greater than 15 seconds); retinal arterial or venous occlusion (any deviation from baseline); or diffuse retinal permeability alteration effecting retinal circulation in the absence of intraocular inflammation

Systemic DLT:

Grade III (severe) or IV (life-threatening) toxicities, or any significant severe toxicity deemed related to study drug by the investigator.

Selection Criteria.

Patients for the study were selected using the following inclusion and exclusion criteria:

Inclusion Criteria: The ophthalmic criteria included best corrected visual acuity in the study eye worse than 20/100 on the ETDRS chart, best corrected visual acuity in the fellow eye equal to or better than 20/400, subfoveal choroidal neovascularization with active CNV (either classic and/or occult) of less than 12 total disc areas in size secondary to age related macular degeneration, clear ocular media and adequate pupillary dilatation to permit good quality stereoscopic fundus photography, and intraocular pressure of 21 mmHg or less. General criteria included patients of either sex, aged ≧50 years; performance Status ≦2 according to the Eastern Cooperative Oncology Group (ECOG)/World Health Organization (WHO) scale, normal electrocardiogram (ECG) or clinically non-significant changes; women must be using an effective contraceptive, be post-menopausal for at least 12 months prior to study entry, or surgically sterile; if not, a serum pregnancy test must be performed within 48 hours prior to treatment and the result made available prior to treatment initiation, an effective form of contraceptive should be implemented for at least 28 days following the last dose of EYE001; adequate hematological function: hemoglobin ≧10 g/dl; platelet count ≧150×10⁹/l; WBC ≧4×10⁹/l; PTT within normal range of institution; adequate renal function: serum creatinine and BUN within 2× the upper limit of normal (ULN) institution; adequate liver function: serum bilirubin ≦1.5 mg/dl; SGOT/ALT, SGPT/AST, and alkaline phosphatase within 2×ULN of institution; written informed consent; and ability to return for all study visits.

Exclusion Criteria: Patients were not eligible for the study if any of the following criteria were present in the study eye or systemically: patients scheduled to receive, or have received any prior Photodynamic Therapy with Visudyne; significant media opacities, including cataract, which might interfere with visual acuity, assessment of toxicity or fundus photography; presence of other causes of choroidal neovascularization, including pathologic myopia (spherical equivalent of −8 diopters or more negative), the ocular histoplasmosis syndrome, angioid streaks, choroidal rupture and multifocal choroiditis; patients in whom additional laser treatment for choroidal neovascularization might be indicated or considered; any intraocular surgery within 3 months of study entry; previous vitrectomy; previous or concomitant therapy with another investigational agent to treat AMD except multivitamins and trace minerals; previous radiation to the fellow eye with photons or protons; known allergies to the fluorescein dye used in angiography or to the components of EYE001 formulation; any of the following underlying systemic diseases including: uncontrolled diabetes mellitus or presence of diabetic retinopathy, cardiac disease: myocardial infarction within 12 months prior to study entry, and/or coronary disease associated with clinical symptoms, and/or indications of ischemia noted on ECG, impaired renal or hepatic function, stroke (within 12 months of study entry), active infection, active bleeding disorders, any major surgical procedure within one month of study entry, active peptic ulcer disease with bleeding within 6 months of study entry; concomitant systemic therapy with corticosteroids (e.g. oral prednisone), or other anti-angiogenic drugs (e.g. thalidomide); previous radiation to the head and neck; any treatment with an investigational agent in the past 60 days for any condition; any diagnosis of cancer in the past 5 years, with the exception of basal or squamous cell carcinoma.

Study Medication.

Drug Supply

EYE001 was used as the anti-VEGF therapy in this study. EYE001 drug substance is a pegylated anti-VEGF aptamer. It was formulated in phosphate buffered saline at pH 5-7. Sodium hydroxide or hydrochloric acid may be added for pH adjustment.

EYE001 was formulated at three different concentrations: 3 mg/100 ul, 2 mg/100 ul and 1 mg/100 ul packaged in a sterile 1 ml, USP Type I graduated glass syringe fitted with a sterile 27-gauge needle. The drug product was preservative-free and intended for single use by intravitreous injection only. The product was not used if cloudy or particles were present.

The active ingredient was EYE001 Drug Substance, (Pegylated) anti-VEGF aptamer, and 30 mg/ml, 20 mg/ml and 10 mg/ml concentrations. The excipients were Sodium Chloride, USP; Sodium Phosphate Monobasic, Monohydrate, USP; Sodium Phosphate Dibasic, Heptahydrate, USP; Sodium Hydroxide, USP; Hydrochloric acid, USP; and Water for injection, USP.

Dose and Administration

Preparation. The drug product was a ready-to-use sterile solution provided in a single-use glass syringe. The syringe was removed from refrigerated storage at least 30 minutes (but not longer than 4 hours) prior to use to allow the solution to reach room temperature. Administration of the syringe contents involved attaching the threaded plastic plunger rod to the rubber stopper inside the barrel of the syringe. The rubber end cap was then removed to allow administration of the product.

Treatment Regimen and Duration. EYE001 was administered as a 100 μl intravitreal injections on three occasions at 28 day intervals. Patients were enrolled to receive 3 mg/injection. If 3 or more patients experienced Dose-Limiting Toxicity (DLT's), the dose was reduced to 2 mg and further to 1 mg, if necessary, each in an additional 10 patients.

PDT Administration.

PDT was given with EYE001 only in cases with predominantly classic CNV. The standard requirements and procedures for PDT administration were used as described in Arch Ophthalmol 1999, 117:1329-1345. PDT was required to be given 5-10 days prior to administration of the anti-VEGF aptamer.

Patient Enrollment.

Before recruitment of patients into the study, written Institutional Review Board (IRB) approval of the protocol, and informed consent form were obtained. Case report form screening pages were completed by study site personnel. Patients who meet the eligibility criteria and have provided written informed consent were enrolled in the study.

Follow-up Schedule.

Patients were clinically evaluated by the ophthalmologist several days after injection and again one-month later just prior to the next injection. ETDRS visual acuities, kodachrome photography and fluorescein angiography were performed monthly for the first 4 months.

Endpoints.

The safety parameters given under the DLT section above were the primary endpoint of the studies. In addition, the percentage of patients with stabilized (0 line change or better) or improved vision at 3 months, the percentage of patients with a 3-line or greater improvement at 3 months, and the need for PDT re-treatment at 3 month as determined by the investigator were other endpoints studied.

Results.

No serious related adverse events were noted for the 21 patients treated in this study. Two patients experienced serious unrelated adverse events. One patient, an 86 year-old woman with a long-standing history of peripheral vascular disease as well as borderline hypertension and type II diabetes mellitus experienced 2 myocardial infarctions, the second of which was fatal. The first event occurred 11 days following the first intraocular injection of anti-VEGF aptamer. The second event occurred 16 days following the third and last injection. The acute myocardial infarctions took place approximately 2 months apart. These events were believed to be unrelated to aptamer therapy by the investigator and systemic levels of the drug are negligible based on pharmacokinetic data. A second patient, a 76 year-old man with a 10-month history of depression attempted suicide with ingestion of acetaminophen 11 days after the third and last dose of anti-VEGF aptamer. The patient's mental condition improved. Treatment of the patient has remained unchanged and the patient is presently followed in the study.

Tables 1A-C show the unrelated or non-severe events reported in these groups. In patients treated with the anti-VEGF aptamer alone, ocular adverse events probably associated with administration of the anti-VEGF aptamer included vitreous floaters (4 Events), mild anterior chamber inflammation (3 Events), ocular irritation (2 Events), increased intraocular pressure (1 Event), intraocular air (1 Event), vitreous haze (1 Event), subconjunctival hemorrhage (1 Event), eye pain (1 Event), lid edema/erythema (1 Event), dry eye (1 Event) and conjunctival injection (1 Event). Events possibly related to administration of anti-VEGF aptamer included, asteroid hyalosis (1 Event), abnormal vision (1 Event) and fatigue (1 Event). Events termed unrelated to administration of anti-VEGF aptamer included headache (1 Event) and weakness (1 Event). In patients treated with the anti-VEGF aptamer and PDT adverse events probably associated with this combination of therapy included ptosis (5 Events), mild anterior chamber inflammation (4 Events), corneal abrasion (3 Events), eye pain (3 Events), foreign body sensation (2 Events), chemosis (1 Event), subconjunctival hemorrhage (1 Event) and vitreous prolapse (1 Event). Events possibly related to combination therapy included fatigue (2 Events). Events unrelated to combination therapy included pigment epithelial detachment (1 Event), joint pain (1 Event), upper respiratory infection (1 Event) and bladder infection (1 Event). The increase in ptosis and corneal abrasion seen in the setting of combination therapy may be related to the use of a contact lens in association with PDT. Of note, all instances of anterior chamber inflammation or vitreous haze were mild and transient in nature.

TABLE 1A
Adverse events associated with administration of anti-VEGF
aptamer alone or in combination with PDT.

| | | | Anti-VEGF | |
|---|---|---|---|---|
| | Anti-VEGF Aptamer | | Aptamer & PDT | |
| | N (%) | | N (%) | |
| Adverse Event | 10 Patients | | 11 Patients | |
| Ptosis | 0 | | 5 (45.4) | |
| Lid Edema/Erythema | 1 (10) | | 2 (18.2) | |
| Conjunctival Injection | 1 (10) | | 0 | |
| Chemosis | 0 | | 1 (9.1) | |
| Subconjunctival | 1 (10) | | 1 (9.1) | |
| Hemorrhage | | | | |
| Dry Eye | 1 (10) | | 0 | |
| Corneal Abrasion | 0 | | 3 (27.3) | |
| Anterior Chamber | 3 (30) | 1+ Cells | 4 (36.4) | Trace |
| Inflammation | | | | Cells |
| | | Trace Cells | | 1+ KP; |
| | | | | Trace |
| | | | | Cells |
| | | Trace Cells | | Trace |
| | | | | Cells |
| | | | | Trace |
| | | | | Cells |

| | | |
|---|---|---|
| IOP Increase | 1 (10) | 0 |
| Pupillary Abnormalities | 0 | 0 |
| Rubeosis | 0 | 1 (9.1) |
| Cataract | 0 | 0 |
| vitreous Haze | 1 (10) | 2 (18.2) |
| Vitreous Prolapse | 0 | 1 (9.1) |
| Vitreous Floaters | 4 (40) | 0 |
| Asteroid Hyalosis | 1 (10) | 0 |
| Intraocular Air | 1 (10) | 0 |
| Peripapillary Hemorrhage | 0 | 1 (9.1) |
| Pigment Epithelial | 0 | 1 (9.1) |
| Detachment | | |
| Abnormal Vision | 1 (10) | 0 |
| Photopsia | 1 (10) | 0 |
| Foreign Body Sensation | 1 (10) | 2 (18.2) |
| Eye Pain | 1 (10) | 3 (27.3) |
| Blepharospasm | 0 | 1 (9.1) |
| Ocular Irritation | 2 (20) | 1 (9.1) |
| Ocular Tenderness | 0 | 1 (9.1) |
| Ocular Pruritis | 1 (10) | 0 |
| Tearing | 1 (10) | 0 |
| Headache | 1 (10) | 0 |
| Rhinorrhea | 0 | 1 (9.1) |
| Fatigue | 1 (10) | 2 (18.2) |
| Weakness | 1 (10) | 0 |
| Joint Pain | 0 | 1 (9.1) |
| Upper Respiratory Infection | 0 | 1 (9.1) |
| Bladder Infection | 0 | 1 (9.1) |

TABLE 1B
Adverse events associated with administration of anti-VEGF aptamer
alone.

| | Anti-VEGF Aptamer N |
|---|---|
| Adverse Event Relationship | 10 Patients |
| Probably: | |
| Vitreous Floaters | 4 |
| Anterior Chamber Inflammation | 3 |
| Ocular Irritation | 2 |
| Vitreous Haze | 1 |
| Increased Intraocular Pressure | 1 |
| Intraocular Air | 1 |
| Subconjunctival Hemorrhage | 1 |
| Conjunctival Injection | 1 |
| Eye Pain | 1 |
| Lid Edema/Erythema | 1 |
| Dry Eye | 1 |
| Possibly: | |
| Asteroid Hyalosis | 1 |
| Abnormal Vision | 1 |
| Fatigue | 1 |
| Unrelated: | |
| Headache | 1 |
| Weakness | 1 |

TABLE 1C
Adverse events associated with administration of anti-VEGF aptamer
and PDT.

| | Anti-VEGF Aptamer & |
|---|---|
| Adverse Event Relationship | PDT N 11 Patients |
| Probably: | |
| Ptosis | 5 |
| Anterior Chamber Inflammation | 4 |
| Corneal Abrasion | 3 |
| Eye Pain | 3 |
| Foreign Body Sensation | 2 |
| Chemosis | 1 |
| Subconjunctival Hemorrhage | 1 |
| Vitreous Prolapse | 1 |
| Possibly: | |
| Fatigue | 2 |
| Unrelated: | |
| Pigment Epithelial Detachment | 1 |
| Joint Pain | 1 |
| Upper Respiratory Infection | 1 |
| Bladder Infection | 1 |

Two patients elected to prematurely terminate their participation in the study. One patient believed that her vision was not improving and did not want further injections. The other patient had depression and transportation problems. Both patients withdrew their consent prior to the third and last injection of the aptamer. Visual acuity in both patients remained stable throughout their participation in the study. A third patient died prior to the final visit.

No dose decrease was required for any patients in the study. Review of color photographs and fluorescein angiograms of these patients revealed no signs of retinal vascular or choroidal toxicity.

Of those patients (N=8) who completed the 3-month treatment regimen of the anti-VEGF aptamer alone 87.5% had stabilized or improved vision and 25.0% had a 3-line improvement of vision on the ETDRS chart at 3 months (See Table 2).

TABLE 2
Visual data of patients with subfoveal CNV treated with anti-
VEGF aptamer alone.

| | | | | | | ±No of |
|---|---|---|---|---|---|---|
| | | | | | | Lines |
| | | | | | | At |
| Patient # | Baseline | Day 8 | Day 29 | Day 57 | Day 85 | Day 85 |
| 03-001 | 20/50 | 20/40 | 20/40 | 20/32 | 20/32 | +2 |
| 04-001 | 20/125 | 20/64 | 20/80 | 20/80 | 20/80 | +2 |
| 06-001 | 20/160 | 20/125 | 20/100 | 20/125 | OUT | +1 |
| 07-001 | 20/100 | 20/100 | 20/64 | 20/80 | 20/80 | +1 |
| 07-002 | 20/320 | 20/80 | 20/64 | 20/64 | 20/50 | +8 |
| 08-001 | 20/125 | 20/125 | 20/100 | 20/100 | 20/160 | −1 |
| 09-001 | 20/500 | 20/200 | 20/400 | 20/320 | OUT | +2 |
| | | | | (Day 36) | | |
| 10-001 | 20/500 | 20/640 | 20/500 | 20/400 | 20/500 | 0 |
| 10-002 | 20/200 | 20/125 | 20/160 | 20/160 | 20/160 | +1 |
| 10-003 | 20/400 | 20/160 | 20/160 | 20/160 | 20/126 | +5 |

CHANGE IN VISION AT 3 MONTHS

| | Stabilized or | ≧3 Line |
|---|---|---|
| | Improved | Improvement |
| EYE001 | 87.5% | 25.0% |
| Treated - (N = 8) | | |
| which represents all | | |
| eyes that completed | | |
| the protocol. | | |

Eleven patients were treated with both the anti-VEGF aptamer and PDT. In this group of patients (N=10) who completed the 3-month treatment regimen, 90% had stabilized or improved vision and 60% showed a 3-line improvement of vision on the ETDRS chart at 3 months (Table 3). These 3-line improvements included gains of +3, +5, +4, +4, +6, and +3 ETDRS lines of vision.

TABLE 3
Visual data of patients with subfoveal CNV treated with anti-
VEGF aptamer combined with PDT.

| | | | | | | | ±No of Lines |
|---|---|---|---|---|---|---|---|
| | | | | | | Repeat | At latest time- |
| Patient # | Baseline | Day 8 | Day 29 | Day 57 | Day 85 | PDT | point |
| 06-011 | 20/400 | 20/320 | 20/100 | 20/640 | 20/200 | NO | +3 |
| 06-012 | 20/250 | 20/160 | 20/125 | 20/125 | 20/80 | NO | +5 |
| 08-011 | 20/40 | 20/32 | 20/20 | 20/20 | 20/26 | YES | +2 |
| 10-011 | 20/160 | 20/160 | 20/160 | 20/160 | OUT | NO | 0 |
| 05-011 | 20/100 | 20/64 | 20/64 | 20/64 | 20/40 | NO | +4 |
| 12-011 | 20/160 | 20/100 | 20/250 | 20/200 | 20/200 | NO | −1 |
| 06-013 | 20/800 | 20/640 | 20/800 | 20/800 | 20/320 | YES | +4 |
| 02-011 | 20/500 | 20/200 | 20/160 | 20/80 | 20/126 | YES | +6 |
| 06-014 | 20/100 | 20/80 | 20/80 | 20/80 | 20/100 | NO | 0 |
| 06-015 | 20/125 | 20/40 | 20/64 | 20/50 | 20/80 | NO | +2 |
| 02-012 | 20/500 | 20/500 | 20/125 | 20/320 | 20/250 | YES | +3 |

CHANGE IN VISION AT 3 MONTHS

| | Stabilized or | ≧3 Line |
|---|---|---|
| | Improved | Improvement |
| EYE001 | 90% | 60% |
| Treated - (N = 10) | | |
| which represents | | |
| all eyes that | | |
| completed the | | |
| protocol. | | |

Of the remaining patients who did not show a 3-line gain, only one showed a loss of vision at 3 months and this patient lost only one line of vision at this time point. No patient in this group lost more than one line of vision at 3 months.

Repeat PDT treatment at 3 months (whose need was solely determined by the investigator) was performed in 4 of 10 eyes (40%) that participated for the complete duration of the study.

### Other Embodiments

Although the present invention has been described with reference to preferred embodiments, one skilled in the art can easily ascertain its essential characteristics and without departing from the spirit and scope thereof, can make various changes and modifications of the invention to adapt it to various usages and conditions. Those skilled in the art will recognize or be able to ascertain using no more than routine experimentation, many equivalents to the specific embodiments of the invention described herein. Such equivalents are intended to be encompassed in the scope of the present invention.

All publications, patents, and patent applications mentioned in this specification are herein incorporated by reference.

We claim:

1. A method for treating an ocular neovascular disease comprising administering a therapeutically effective amount of a formulation comprising an anti-VEGF aptamer conjugated to a polyethylene glycol, wherein the formulation comprises the aptamer at a concentration of 1 to 30 mg/ml in a pharmaceutically acceptable carrier formulation for local administration into the eye, wherein the aptamer is 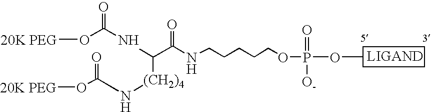
Ligand Component=fCmGmGrArAfUfCmAmGfUmGmAmAfUmGfCfUfUmAfUmAfCmAfUfCfCmG-3′3′-(VEGF ligand)

2. A method according to claim 1 wherein the ocular neovascular disease is selected from the group consisting of ischemic retinopathy, intraocular neovascularization, macular degeneration, corneal neovascularization, retinal neovascularization, choroidal neovascularization, diabetic macular edema, diabetic retinal ischemia, diabetic retinal edema, and proliferative diabetic retinopathy.

3. A method according to claim 1 wherein the ocular neovascular disease is selected from the group consisting of diabetic ischemic retinopathy, diabetic retinal ischemia, and proliferative diabetic retinopathy.

4. A method according to claim 1 wherein the aptamer is administered by intravitreous injection.

5. A method according to claim 1 wherein the aptamer is administered every 4-6 weeks.

6. A method according to claim 4 wherein the treatment is continued for a period of at least one year.

7. A method according to claim 1 wherein the therapeutically effective amount is about 0.1-3.0 mg.

8. A method according to claim 1 wherein the therapeutically effective amount is about 0.1-1.0 mg.

9. A method according to claim 1 wherein the therapeutically effective amount is about 0.3 mg.

10. A method according to claim 1 further comprising administering phototherapy.

11. A method according to claim 1, wherein the carrier comprises sodium phosphate and sodium chloride.

12. A method according to claim 1, wherein the carrier comprises 10 mM sodium phosphate and 0.9% sodium chloride.
